(19) United States
(12) Patent Application Publication
(10) Pub. No.: US 2022/0331410 A1
(43) Pub. Date: Oct. 20, 2022

(54) NEUROTOXIN COMPOSITIONS FOR USE IN TREATING GASTROPARESIS

(71) Applicant: AEON Biopharma, Inc., Irvine, CA (US)

(72) Inventors: Gregory F. Brooks, Irvine, CA (US); Eric Carter, Laguna Beach, CA (US)

(21) Appl. No.: 17/641,039

(22) PCT Filed: Sep. 9, 2020
(86) PCT No.: PCT/US2020/049959

§ 371 Date: Mar. 7, 2022

Related U.S. Application Data
(60) Provisional application No. 62/950,794, filed on Dec. 19, 2019.
(60) Provisional application No. 62/897,520, filed on Sep. 9, 2019.

(51) Int. Cl. A61K 38/48; A61P 1/14; A61K 9/00

(52) U.S. Cl. CPC A61K 38/4893; A61P 1/14; A61K 9/0019

(57) ABSTRACT

Disclosed herein are compositions and methods for use in treating gastroparesis.

### FIELD

The present specification relates to the use of neurotoxins administered to specific locations in the digestive tract, for example to treat gastroparesis.

### BACKGROUND

The primary function of the stomach is the temporary storage of ingested food and fluids, together with the preparation of chyme (the pulpy acidic fluid which passes from the stomach to the small intestine, consisting of gastric juices and partly digested food) through physical mixing and chemical action and the controlled release of aliquots of chyme into the duodenum. This initial process of digestion of nutrients and fluids involves the contraction and relaxation of overlapping muscle layers of the stomach, as well as the contraction and relaxation of the pyloric sphincter, and is coordinated via the central nervous system.

Gastroparesis is a complex, debilitating gastric motility disorder with its sufferers facing challenging symptom management. A diagnosis of gastroparesis is based on a documented history of gastroparesis symptoms and signs, together with objectively delayed gastric emptying in the absence of mechanical obstruction or other etiologies. An increasing body of evidence suggests that gastroparesis is also associated with hypertonicity of the fundus and proximal corpus of the stomach, resulting in a failure of receptive relaxation. In idiopathic or disease-related gastroparesis, the dysfunction of one or more of these physiologic processes leads to an interrelated symptom complex, including nausea in over 90% of patients, vomiting in 70-90% of patients, and upper abdominal pain in up to 85% of patients.

Thus, a hypertonic pyloric sphincter or proximal duodenum may impair the ordered emptying of chyme from the stomach resulting in impaired gastric emptying and provides a rationale for targeting injections in this area. When associated with a failure of relaxation and accommodation of the fundus and cardia of the stomach, impaired gastric emptying may further accentuate early satiety, postprandial fullness, nausea, vomiting and upper abdominal discomfort and pain.

Given the limited efficacy of current treatment options and associated serious side effects, significant research continues to identify alternative therapeutic options for the safe and effective treatment of gastroparesis. Further research and novel treatment options are needed to address the substantial unmet medical needs of gastroparesis.

Botulinum toxin injection has been shown to be effective in treating disorders of smooth muscle hypertonicity in the gastrointestinal tract. Endoscopic intra-sphincteric injection of the pylorus with botulinum toxin improves symptoms, signs and gastric emptying in patients with gastroparesis. Botulinum toxin may interfere with hypertonic contractions of the pylorus, resulting in partial relaxation and this may be the reason for the improvement of gastric stasis after botulinum toxin injection. It is already used for the prevention and treatment of gastric stasis as a complication of esophagectomy associated with vagotomies, oropharyngeal dysphagia, diffuse esophageal spasm, achalasia, refractory gastroparesis and anal fissures.

Patients with idiopathic or diabetic gastroparesis who are refractory to medical therapy have shown improvement in symptoms and signs with botulinum toxin injection therapy. The improvement can be maintained for a duration of approximately 6-8 weeks. Patients who had a positive response to the first dose usually continue to respond to repeat injections, and retreatment is often required.

However, all of these treatments suffer from inconsistent effectiveness as well as short duration of effect. To improve this outcome, successfully treating gastroparesis patients may require an increased neurotoxin dose administered into more injection sites as compared to current methods.

### SUMMARY

Disclosed herein are compositions and methods comprising neurotoxins, for example Clostridial neurotoxins including botulinum toxins, and the use thereof to treat gastroparesis, via targeted endoscopic intra-sphincteric injection of the pylorus and/or the fundus and proximal corpus to treat and prevent the recurrence of the signs and symptoms of gastroparesis, and improve quantitative measures of gastric emptying in patients with gastroparesis. The failure or reduction of stomach muscle relaxation provides the rationale for targeting injections in the proximal stomach. These interrelated common signs and symptoms of gastroparesis may be further aggravated by antral dysmotility or uncoordinated peristalsis resulting in the failure of antral mixing and upper abdominal pain thus providing the rational for injections in the pyloric antrum, also known as the gastric antrum.

Disclosed methods comprise an injection paradigm localizing and isolating injection sites circumferentially in the pyloric sphincter, and injection sites on the fundus and proximal corpus on the greater curvature of the stomach, administered approximately one centimeter apart, providing a therapeutically effective amount of a neurotoxin to the isolated smooth muscle that controls both the valve-like function of the pyloric sphincter allowing solid food and liquids to pass from the stomach in a controlled manner and the controlled relaxation of the fundus in response to a meal preventing early satiety and other cardinal signs and symptoms of gastroparesis.

Disclosed methods further comprise methods of treating gastroparesis comprising the steps of administering a first botulinum toxin dose into the pyloric sphincter; administering a second botulinum toxin dose in the pyloric region, including the pyloric canal and/or the pyloric antrum; and administering a third botulinum toxin dose into the fundus and proximal corpus; thereby reducing the severity of at least one or more symptoms of the disorder including abdominal pain or discomfort, episodic stomach swelling, stomach pain, early fullness, abdominal bloating, early satiety, post-prandial fullness, post-prandial satiety, nausea, vomiting, acid reflux, diarrhea, problems swallowing, and combinations thereof.

Disclosed methods further comprise methods of treating gastroparesis comprising administering a first botulinum toxin dose into the pyloric sphincter; administering a second botulinum toxin dose in the pyloric region including the pyloric canal and/or the pyloric antrum; and administering a third botulinum toxin dose into the duodenum; thereby reducing the severity of at least one or more symptoms of the disorder including abdominal pain or discomfort, episodic stomach swelling, stomach pain, early fullness, abdominal bloating, early satiety, post-prandial fullness, post-prandial satiety, nausea, vomiting, acid reflux, diarrhea, problems swallowing, and combinations thereof.

Disclosed methods further comprise methods of treating gastroparesis comprising sequentially;
a. administering a botulinum toxin dose into the pyloric sphincter;
b. administering a botulinum toxin dose into the pyloric canal;
c. administering a botulinum toxin dose into the duodenum (to aid in chemical digestion of and preparation for absorption in the small intestine) during the initial or follow-up treatment; and
d. optionally injecting a botulinum toxin dose into the fundus, proximal corpus and/or the pyloric antrum, thereby reducing the severity of at least one or more symptoms of the disorder including abdominal pain or discomfort, episodic stomach swelling, stomach pain, early fullness, abdominal bloating, early satiety, post-prandial fullness, post-prandial satiety, nausea, vomiting, acid reflux, diarrhea, problems swallowing, and combinations thereof.

### BRIEF DESCRIPTION OF THE DRAWING

FIG. 1 shows the stomach and relevant structures therein, including the location of the pyloric sphincter and fundus. The proximal corpus is located just “below” the fundus.

FIG. 2 shows the pathophysiology of gastroparesis and impact on gastric function in diabetic gastroparesis.

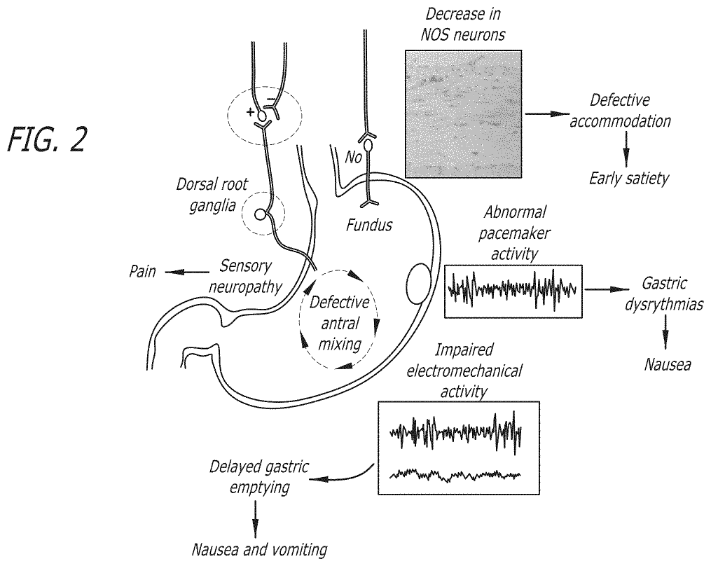

FIG. 3 shows the pathophysiology of gastroparesis and impact on gastric function in diabetic gastroparesis.

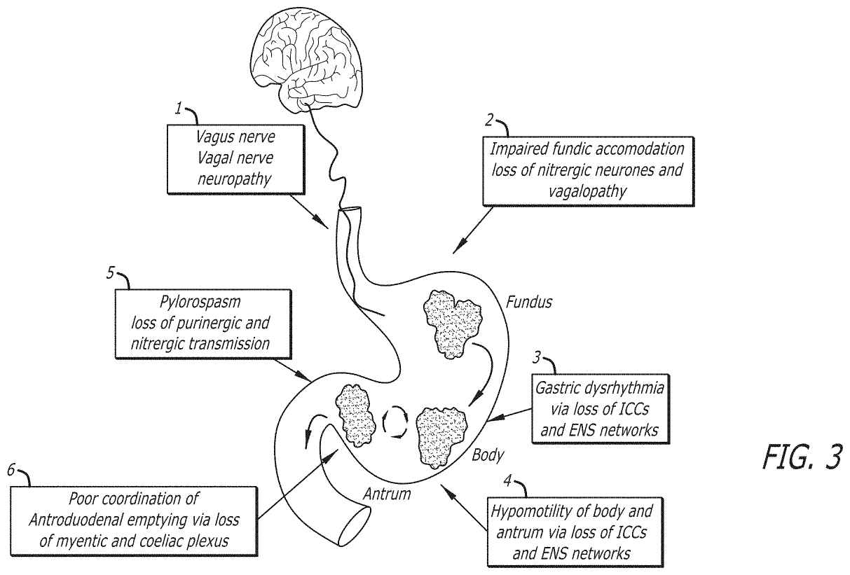

### DETAILED DESCRIPTION

The present disclosure is directed toward methods for reducing the occurrence and severity of symptoms associated with, gastroparesis.

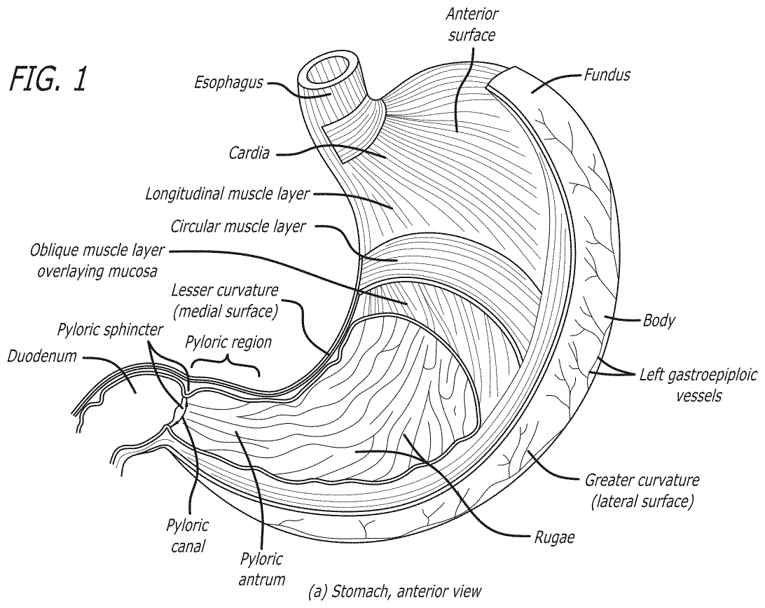

Disclosed methods comprise, for example, an injection paradigm localizing and isolating, for example, 5-7 injection sites circumferentially in the pyloric sphincter, and 10-30 injection sites on the fundus and proximal corpus on the greater curvature of the stomach (the pyloric sphincter and fundus are shown in FIG. 1) administered approximately one centimeter apart, and 5-30 injection sites on the pyloric antrum, pyloric canal, and duodenum, providing a therapeutically effective amount of a neurotoxin to the isolated smooth muscle that controls both the valve-like function of the pyloric sphincter allowing solid food and liquids to pass from the stomach in a controlled manner and the controlled relaxation of the fundus in response to a meal preventing early satiety and other cardinal signs and symptoms of gastroparesis. The dilution of the neurotoxin can be, for example, 4 to 8 mL per 200 Units (U) or 2 to 4 mL per 100 U, and the depth of each injection can be, for example, 2-5 mm into the smooth muscle, applied in a perpendicular manner using, for example, a 23 to 27 gauge sclerotherapy or similar needle with a tip length of 2-5 mm advanced through an endoscope in order to maximize the therapeutic effect and ensure safety. The patient's sensitivity to and tolerance of the neurotoxin can be determined in the initial treatment by administering a total dose of, for example, 50 to 200 U to target sites in the pyloric sphincter and 50 to 200 U to target sites in the gastric fundus and proximal corpus to maximize the efficacy and safety of the neurotoxin.

### Definitions

“Administration,” or “to administer” means the step of giving (i.e. administering) a pharmaceutical composition or active ingredient to a subject. The pharmaceutical compositions disclosed herein can be administered via a number of appropriate routs, including oral and intramuscular or subcutaneous routes of administration, such as by injection, topically, or use of an implant.

“Botulinum toxin” or “botulinum neurotoxin” means a neurotoxin derived from Clostridium botulinum, as well as modified, recombinant, hybrid and chimeric botulinum toxins. A recombinant botulinum toxin can have the light chain and/or the heavy chain thereof made recombinantly by a non-Clostridial species. “Botulinum toxin,” as used herein, encompasses the botulinum toxin serotypes A, B, C, D, E, F, G and H. “Botulinum toxin,” as used herein, also encompasses both a botulinum toxin complex (i.e. the 300, 600 and 900 kDa complexes) as well as pure botulinum toxin (i.e. the about 150 kDa neurotoxic molecule), all of which are useful in the practice of the disclosed embodiments.

“Clostridial neurotoxin” means a neurotoxin produced from, or native to, a Clostridial bacterium, such as Clostridium botulinum, Clostridium butyricum or Clostridium beratti, as well as a Clostridial neurotoxin made recombinantly by a Clostridial or non-Clostridial species.

“Fast-acting neurotoxin” as used herein refers to a botulinum toxin that produces effects in the patient more rapidly than those produced by, for example, a botulinum neurotoxin type A. For example, the effects of a fast-acting botulinum toxin (such as botulinum type E) can be produced within 36 hours.

“Fast-recovery neurotoxin” as used herein refers to a botulinum toxin that whose effects diminish in the patient more rapidly than those produced by, for example, a botulinum neurotoxin type A. For example, the effects of a fast-recovery botulinum toxin (such as botulinum type E) can diminish within, for example, 120 hours, 150 hours, 300 hours, 350 hours, 400 hours, 500 hours, 600 hours, 700 hours, 800 hours, or the like. It is known that botulinum toxin type A can have an efficacy for up to 12 months, and in some circumstances for as long as 27 months, when used to treat glands, such as in the treatment of hyperhidrosis. However, the usual duration of an intramuscular injection of a botulinum neurotoxin type A is typically about 3 to 4 months.

“Neurotoxin” means a biologically active molecule with a specific affinity for a neuronal cell surface receptor. Neurotoxin includes Clostridial toxins both as pure toxin and as complexed with one or more non-toxin, toxin-associated proteins.

“Patient” means a human or non-human subject receiving medical or veterinary care.

“Pharmaceutical composition” means a formulation in which an active ingredient can be a Clostridial toxin. The word “formulation” means that there is at least one additional ingredient (such as, for example and not limited to, an albumin [such as a human serum albumin or a recombinant human albumin] and/or sodium chloride) in the pharmaceutical composition in addition to a Clostridial (for example, a botulinum neurotoxin) active ingredient. A pharmaceutical composition is therefore a formulation which is suitable for diagnostic, therapeutic or cosmetic administration to a subject, such as a human patient. The pharmaceutical composition can be in a lyophilized or vacuum dried condition, a solution formed after reconstitution of the lyophilized or vacuum dried pharmaceutical composition with saline or water, for example, or as a solution that does not require reconstitution. As stated, a pharmaceutical composition can be liquid, semi-solid, or solid. A pharmaceutical composition can be animal-protein free.

“Purified botulinum toxin” means a pure botulinum toxin or a botulinum toxin complex that is isolated, or substantially isolated, from other proteins and impurities which can accompany the botulinum toxin as it is obtained from a culture or fermentation process. Thus, a purified botulinum toxin can have at least 95%, and more preferably at least 99% of the non-botulinum toxin proteins and impurities removed.

“Therapeutic formulation” means a formulation that can be used to treat and thereby alleviate a disorder or a disease and/or symptom associated thereof.

“Therapeutically effective amount” means the level, amount or concentration of an agent (e.g. such as a Clostridial toxin or pharmaceutical composition comprising clostridial toxin) needed to treat a disease, disorder or condition without causing significant negative or adverse side effects.

“Treat,” “treating,” or “treatment” means an alleviation or a reduction (which includes some reduction, a significant reduction, a near total reduction, and a total reduction), resolution, or prevention (temporarily or permanently) of a symptom, disease, disorder or condition, so as to achieve a desired therapeutic or cosmetic result, such as by healing of injured or damaged tissue, or by altering, changing, enhancing, improving, ameliorating and/or beautifying an existing or perceived disease, disorder or condition.

“Unit” or “U” means an amount of active botulinum neurotoxin standardized to have equivalent neuromuscular blocking effect as a Unit of commercially available botulinum neurotoxin type A (for example, Onabotulinumtoxin A (BOTOX®)).

Neurotoxin Compositions

Embodiments disclosed herein comprise neurotoxin compositions. Such neurotoxins can be formulated in any pharmaceutically acceptable formulation in any pharmaceutically acceptable form. The neurotoxin can also be used in any pharmaceutically acceptable form supplied by any manufacturer. Disclosed embodiments comprise use of Clostridial neurotoxins.

The Clostridial neurotoxin can be made by a Clostridial bacterium, such as by a Clostridium botulinum, Clostridium butyricum, or Clostridium beratti bacterium. Additionally, the neurotoxin can be a modified neurotoxin; that is a neurotoxin that has at least one of its amino acids deleted, modified or replaced, as compared to the native or wild type neurotoxin. Furthermore, the neurotoxin can be a recombinantly produced neurotoxin or a derivative or fragment thereof.

In disclosed embodiments, the neurotoxin is formulated in unit dosage form; for example, it can be provided as a sterile solution in a vial or as a vial or sachet containing a lyophilized powder for reconstituting in a suitable vehicle, such as saline for injection.

In embodiments the neurotoxin, for example botulinum toxin, is formulated in a solution containing saline and pasteurized Human Serum Albumin (HSA), which stabilizes the toxin and minimizes loss through non-specific adsorption. The solution can be sterile filtered (0.2 μm filter), filled into individual vials, and then vacuum-dried to give a sterile lyophilized powder. In use, the powder can be reconstituted by the addition, for example, of sterile unpreserved normal saline (sodium chloride 0.9% for injection).

In an embodiment, botulinum type A is supplied in a sterile solution for injection with a 5-mL vial nominal concentration of 20 ng/mL in 0.03 M sodium phosphate, 0.12 M sodium chloride, and 1 mg/mL HSA, at pH 6.0.

Although the composition may only contain a single type of neurotoxin, for example botulinum type A, disclosed compositions can include two or more types of neurotoxins, which can provide enhanced therapeutic effects in treating the disorders. For example, a composition administered to a patient can include botulinum types A and E, or A and B, or the like. Administering a single composition containing two different neurotoxins can permit the effective concentration of each of the neurotoxins to be lower than if a single neurotoxin is administered to the patient while still achieving the desired therapeutic effects. This type of “combination” composition can also provide benefits of both neurotoxins, for example, quicker effect combined with longer duration.

The composition administered to the patient can also contain other pharmaceutically active ingredients, such as, protein receptor or ion channel modulators, in combination with the neurotoxin or neurotoxins. These modulators may contribute to the reduction in neurotransmission between the various neurons. For example, a composition may contain gamma aminobutyric acid (GABA) type A receptor modulators that enhance the inhibitory effects mediated by the GABA_{A} receptor. The GABA_{A} receptor inhibits neuronal activity by effectively shunting current flow across the cell membrane. GABA_{A} receptor modulators may enhance the inhibitory effects of the GABA_{A} receptor and reduce electrical or chemical signal transmission from the neurons. Examples of GABA_{A} receptor modulators include benzodiazepines, such as diazepam, oxaxepam, lorazepam, prazepam, alprazolam, halazeapam, chordiazepoxide, and chlorazepate. Compositions may also contain glutamate receptor modulators that decrease the excitatory effects mediated by glutamate receptors. Examples of glutamate receptor modulators include agents that inhibit current flux through AMPA, NMDA, and/or kainate types of glutamate receptors. Further disclosed compositions comprise esketamine.

Disclosed neurotoxin compositions can be injected into the patient using a needle or a needleless device. In certain embodiments, the method comprises sub-dermally injecting the composition in the individual. For example, administering may comprise injecting the composition through a needle of, in embodiments, no greater than about 30 gauge. In embodiments, the injection should be made in a perpendicular manner using a 23 to 27 gauge sclerotherapy or similar needle with a tip length of, for example, 2-5 mm. In certain embodiments, the method comprises administering a composition comprising a botulinum toxin, for example botulinum toxin type A.

Administration of the disclosed compositions can be carried out by syringes, catheters, needles and other means for injecting. The injection can be performed on any area of the mammal's body that is in need of treatment, however disclosed embodiments contemplate injection into the patient's stomach and the vicinity thereof. The injection can be into any specific area such as epidermis, dermis, fat, smooth or skeletal muscle, nerve junction, or subcutaneous layer.

More than one injection and/or sites of injection may be necessary to achieve the desired result. Also, some injections, depending on the location to be injected, may require the use of fine, hollow, Teflon®-coated needles. In certain embodiments, guided injection is employed, for example by electromyography, or ultrasound, or fluoroscopic guidance or the like.

The frequency and the amount of injection under the disclosed methods can be determined based on the nature and location of the particular area being treated. In certain cases, however, repeated injection may be desired to achieve optimal results. The frequency and the amount of the injection for each particular case can be determined by the person of ordinary skill in the art.

Although examples of routes of administration and dosages are provided, the appropriate route of administration and dosage are generally determined on a case by case basis by the attending physician. For example, the route and dosage for administration of a Clostridial neurotoxin according to the present disclosed invention can be selected based upon criteria such as the solubility characteristics of the neurotoxin chosen as well as the intensity and scope of the condition being treated.

Methods of Use

Methods disclosed herein can comprise administration of a neurotoxin, for example a Clostridial toxin, for example a botulinum toxin such as type A, to a patient to prevent or alleviate the symptoms associated with gastroparesis. For example, disclosed methods can comprise prevention or reduction in symptoms comprising, for example, swollen stomach, gassiness, chronic abdominal pain or discomfort, episodic stomach swelling, stomach pain, early fullness, abdominal bloating, early satiety, post-prandial fullness, post-prandial satiety, nausea, vomiting, acid reflux, diarrhea, problems swallowing, and combinations thereof.

Disclosed embodiments comprise methods of demonstrating improvement in gastric emptying and reduction in pyloric hypertonicity demonstrated on antro-pyloric manometry in patients with gastroparesis.

Disclosed methods comprise, for example, treating a patient suffering from a gastroparesis with an injection paradigm localizing and isolating injection sites circumferentially, for example in the pyloric sphincter. Disclosed embodiments can comprise multiple injections, for example, between 1 and 5 injection sites, between 5 and 10 injection sites, between 5 and 15 injection sites, between 10 and 20 injection sites, or the like. In embodiments, the number of pyloric sphincter injection sites can comprise, for example, between 3 and 5 injection sites, between 4 and 6 injection sites, between 5 and 7 injection sites, between 6 and 8 injection sites, between 7 and 9 injection sites, between 8 and 10 injection sites, or the like.

In embodiments, the number of pyloric sphincter injection sites can comprise, for example, at least 1 injection site, at least 2 injection sites, at least 3 injection sites, at least 4 injection sites, at least 5 injection sites, at least 6 injection sites, at least 7 injection sites, at least 8 injection sites, at least 9 injection sites, at least 10 injection sites, or the like.

In embodiments, the number of pyloric sphincter injection sites can comprise, for example, not more than 1 injection site, not more than 2 injection sites, not more than 3 injection sites, not more than 4 injection sites, not more than 5 injection sites, not more than 6 injection sites, not more than 7 injection sites, not more than 8 injection sites, not more than 9 injection sites, not more than 10 injection sites, or the like.

In embodiments, the injections to the pyloric sphincter are spaced, for example, 2 mm apart, 3 mm apart, 4 mm apart, 5 mm apart, 6 mm apart, 7 mm apart, 8 mm apart, 9 mm apart, 10 mm apart, 11 mm apart, 12 mm apart, 13 mm apart, 14 mm apart, 15 mm apart, 20 mm apart, 25 mm apart, 30 mm apart, or the like.

In embodiments, the injections to the pyloric sphincter are spaced, for example, at least 2 mm apart, at least 3 mm apart, at least 4 mm apart, at least 5 mm apart, at least 6 mm apart, at least 7 mm apart, at least 8 mm apart, at least 9 mm apart, at least 10 mm apart, at least 15 mm apart, at least 20 mm apart, at least 25 mm apart, at least 30 mm apart, or the like.

In embodiments, the injections to the pyloric sphincter are spaced, for example, not more than 2 mm apart, not more than 3 mm apart, not more than 4 mm apart, not more than 5 mm apart, not more than 6 mm apart, not more than 7 mm apart, not more than 8 mm apart, not more than 9 mm apart, not more than 10 mm apart, not more than 15 mm apart, not more than 20 mm apart, not more than 25 mm apart, not more than 30 mm apart, or the like.

In embodiments, the depth of each pyloric sphincter injection should be, for example, 1 mm, 2 mm, 3 mm, 4 mm, 5 mm, 6 mm, 7 mm, 8 mm, 9 mm, 10 mm, or the like.

In embodiments, the depth of each pyloric sphincter injection should be, for example, between 1 and 4 mm into the smooth muscle of the treatment site, between 2 and 5 mm into the smooth muscle of the treatment site, between 3 and 6 mm into the smooth muscle of the treatment site, between 4 and 7 mm into the smooth muscle of the treatment site. In embodiments, the injection should be made in a perpendicular manner using a 23 to 27 gauge sclerotherapy or similar needle with a tip length of, for example, 2-5 mm, advanced through an endoscope in order to maximize the therapeutic effect and ensure safety.

In embodiments, the spacing between the pyloric sphincter injection sites can be uniform. In embodiments, the spacing between injection sites can be non-uniform, for example the sites can be adjusted to conform with the patient's specific anatomy.

Disclosed embodiments further comprise additional injection sites on the fundus and proximal corpus on the greater curvature of the stomach. For example, disclosed embodiments comprise 1-20 injection sites on the fundus and proximal corpus, 5-25 injection sites on the fundus and proximal corpus, 10-30 injection sites on the fundus and proximal corpus, 15-35 injection sites on the fundus and proximal corpus, 20-40 injection sites on the fundus and proximal corpus, 25-45 injection sites on the fundus and proximal corpus, or the like.

Disclosed embodiments comprise, for example, at least 2 injection sites on the fundus and proximal corpus, at least 4 injection sites on the fundus and proximal corpus, at least 6 injection sites on the fundus and proximal corpus, at least 8 injection sites on the fundus and proximal corpus, at least 12 injection sites on the fundus and proximal corpus, at least 16 injection sites on the fundus and proximal corpus, at least 20 injection sites on the fundus and proximal corpus, at least 24 injection sites on the fundus and proximal corpus, at least 28 injection sites on the fundus and proximal corpus, at least 32 injection sites on the fundus and proximal corpus, at least 36 injection sites on the fundus and proximal corpus, at least 40 injection sites on the fundus and proximal corpus, or the like.

Disclosed embodiments comprise, for example, not more than 4 injection sites on the fundus and proximal corpus, not more than 6 injection sites on the fundus and proximal corpus, not more than 8 injection sites on the fundus and proximal corpus, not more than 12 injection sites on the fundus and proximal corpus, not more than 16 injection sites on the fundus and proximal corpus, not more than 20 injection sites on the fundus and proximal corpus, not more than 24 injection sites on the fundus and proximal corpus, not more than 28 injection sites on the fundus and proximal corpus, not more than 32 injection sites on the fundus and proximal corpus, not more than 36 injection sites on the fundus and proximal corpus, not more than 40 injection sites on the fundus and proximal corpus, or the like.

In embodiments, the injections on the fundus and proximal corpus are spaced, for example, 2 mm apart, 3 mm apart, 4 mm apart, 5 mm apart, 6 mm apart, 7 mm apart, 8 mm apart, 9 mm apart, 10 mm apart, 11 mm apart, 12 mm apart, 13 mm apart, 14 mm apart, 15 mm apart, 20 mm apart, 25 mm apart, 30 mm apart, or the like.

In embodiments, the injections on the fundus and proximal corpus are spaced, for example, at least 2 mm apart, at least 3 mm apart, at least 4 mm apart, at least 5 mm apart, at least 6 mm apart, at least 7 mm apart, at least 8 mm apart, at least 9 mm apart, at least 10 mm apart, at least 15 mm apart, at least 20 mm apart, at least 25 mm apart, at least 30 mm apart, or the like.

In embodiments, the injections on the fundus and proximal corpus are spaced, for example, not more than 2 mm apart, not more than 3 mm apart, not more than 4 mm apart, not more than 5 mm apart, not more than 6 mm apart, not more than 7 mm apart, not more than 8 mm apart, not more than 9 mm apart, not more than 10 mm apart, not more than 15 mm apart, not more than 20 mm apart, not more than 25 mm apart, not more than 30 mm apart, or the like.

In embodiments, the spacing between the fundus and proximal corpus injection sites can be uniform. In embodiments, the spacing between injection sites can be non-uniform, for example the sites can be adjusted to conform with the patient's specific anatomy.

In embodiments, the depth of each fundus and proximal corpus injection should be, for example, 1 mm, 2 mm, 3 mm, 4 mm, 5 mm, 6 mm, 7 mm, 8 mm, 9 mm, 10 mm, or the like.

In embodiments, the depth of each fundus and proximal corpus injection should be, for example, between 1 and 4 mm into the smooth muscle of the treatment site, between 2 and 5 mm into the smooth muscle of the treatment site, between 3 and 6 mm into the smooth muscle of the treatment site, between 4 and 7 mm into the smooth muscle of the treatment site. In embodiments, the injection should be made in a perpendicular manner using a 23 to 27 gauge sclerotherapy or similar needle with a tip length of, for example, 2-5 mm, advanced through an endoscope in order to maximize the therapeutic effect and ensure safety.

Disclosed embodiments further comprise additional injection sites on the pyloric antrum and/or pyloric canal and/or duodenum. For example, disclosed embodiments comprise 1-20 injection sites on the pyloric antrum and/or pyloric canal and/or duodenum, 5-25 injection sites on the pyloric antrum and/or pyloric canal and/or duodenum, 10-30 injection sites on the pyloric antrum and/or pyloric canal and/or duodenum, 15-35 injection sites on the pyloric antrum and/or pyloric canal and/or duodenum, 20-40 injection sites on the pyloric antrum and/or pyloric canal and/or duodenum, 25-45 injection sites on the pyloric antrum and/or pyloric canal and/or duodenum, or the like.

Disclosed embodiments comprise, for example, at least 2 injection sites on the pyloric antrum and/or pyloric canal and/or duodenum, at least 4 injection sites on the pyloric antrum and/or pyloric canal and/or duodenum, at least 6 injection sites on the pyloric antrum and/or pyloric canal and/or duodenum, at least 8 injection sites on the pyloric antrum and/or pyloric canal and/or duodenum, at least 12 injection sites on the pyloric antrum and/or pyloric canal and/or duodenum, at least 16 injection sites on the pyloric antrum and/or pyloric canal and/or duodenum, at least 20 injection sites on the pyloric antrum and/or pyloric canal and/or duodenum, at least 24 injection sites on the pyloric antrum and/or pyloric canal and/or duodenum, at least 28 injection sites on the pyloric antrum and/or pyloric canal and/or duodenum, at least 32 injection sites on the pyloric antrum and/or pyloric canal and/or duodenum, at least 36 injection sites on the pyloric antrum and/or pyloric canal and/or duodenum, at least 40 injection sites on the pyloric antrum and/or pyloric canal and/or duodenum, or the like.

Disclosed embodiments comprise, for example, not more than 4 injection sites on the pyloric antrum and/or pyloric canal and/or duodenum, not more than 6 injection sites on the pyloric antrum and/or pyloric canal and/or duodenum, not more than 8 injection sites on the pyloric antrum and/or pyloric canal and/or duodenum, not more than 12 injection sites on the pyloric antrum and/or pyloric canal and/or duodenum, not more than 16 injection sites on the pyloric antrum and/or pyloric canal and/or duodenum, not more than 20 injection sites on the pyloric antrum and/or pyloric canal and/or duodenum, not more than 24 injection sites on the pyloric antrum and/or pyloric canal and/or duodenum, not more than 28 injection sites on the pyloric antrum and/or pyloric canal and/or duodenum, not more than 32 injection sites on the pyloric antrum and/or pyloric canal and/or duodenum, not more than 36 injection sites on the pyloric antrum and/or pyloric canal and/or duodenum, not more than 40 injection sites on the pyloric antrum and/or pyloric canal and/or duodenum, or the like.

In embodiments, the injections on the pyloric antrum and/or pyloric canal and/or duodenum are spaced, for example, 2 mm apart, 3 mm apart, 4 mm apart, 5 mm apart, 6 mm apart, 7 mm apart, 8 mm apart, 9 mm apart, 10 mm apart, 11 mm apart, 12 mm apart, 13 mm apart, 14 mm apart, 15 mm apart, 20 mm apart, 25 mm apart, 30 mm apart, or the like.

In embodiments, the injections on the pyloric antrum and/or pyloric canal and/or duodenum are spaced, for example, at least 2 mm apart, at least 3 mm apart, at least 4 mm apart, at least 5 mm apart, at least 6 mm apart, at least 7 mm apart, at least 8 mm apart, at least 9 mm apart, at least 10 mm apart, at least 15 mm apart, at least 20 mm apart, at least 25 mm apart, at least 30 mm apart, or the like.

In embodiments, the injections on the pyloric antrum and/or pyloric canal and/or duodenum are spaced, for example, not more than 2 mm apart, not more than 3 mm apart, not more than 4 mm apart, not more than 5 mm apart, not more than 6 mm apart, not more than 7 mm apart, not more than 8 mm apart, not more than 9 mm apart, not more than 10 mm apart, not more than 15 mm apart, not more than 20 mm apart, not more than 25 mm apart, not more than 30 mm apart, or the like.

In embodiments, the spacing between the pyloric antrum and/or pyloric canal and/or duodenum injection sites can be uniform. In embodiments, the spacing between injection sites can be non-uniform, for example the sites can be adjusted to conform with the patient's specific anatomy.

In embodiments, the depth of each pyloric antrum and/or pyloric canal and/or duodenum injection should be, for example, 1 mm, 2 mm, 3 mm, 4 mm, 5 mm, 6 mm, 7 mm, 8 mm, 9 mm, 10 mm, or the like.

In embodiments, the depth of each pyloric antrum and/or pyloric canal and/or duodenum injection should be, for example, between 1 and 4 mm into the smooth muscle of the treatment site, between 2 and 5 mm into the smooth muscle of the treatment site, between 3 and 6 mm into the smooth muscle of the treatment site, between 4 and 7 mm into the smooth muscle of the treatment site. In embodiments, the injection should be made in a perpendicular manner using a 23 to 27 gauge sclerotherapy or similar needle with a tip length of, for example, 2-5 mm, advanced through an endoscope in order to maximize the therapeutic effect and ensure safety.

Disclosed embodiments provide a therapeutically effective amount of a neurotoxin to the isolated smooth muscle that controls both the valve-like function of the pyloric sphincter allowing solid food and liquids to pass from the stomach in a controlled manner, and the controlled relaxation of the fundus in response to a meal, preventing early satiety and other cardinal signs and symptoms of gastroparesis.

In embodiments, the neurotoxin, for example a botulinum type A, can be diluted to, for example, between 1 and 5 mL per 100 U, or between 2 and 4 mL per 100 U, or the like.

The patient's sensitivity to and tolerance of the neurotoxin can be determined in the initial treatment by administering a total dose of 50 to 200 U to target sites in the pyloric sphincter and 50 to 200 U to target sites in the gastric fundus and proximal corpus to maximize the efficacy and safety of the neurotoxin.

Disclosed methods further comprise methods of treating gastroparesis comprising the steps of administering a first botulinum toxin dose into the pyloric sphincter; administering a second botulinum toxin dose in the pyloric region including the pyloric canal and/or the pyloric antrum; and administering a third botulinum toxin dose into the fundus and proximal corpus; thereby reducing the severity of at least one or more symptoms of the disorder including abdominal pain or discomfort, episodic stomach swelling, stomach pain, early fullness, abdominal bloating, early satiety, post-prandial fullness, post-prandial satiety, nausea, vomiting, acid reflux, diarrhea, problems swallowing, and combinations thereof.

Disclosed methods further comprise methods of treating gastroparesis comprising administering a first botulinum toxin dose into the pyloric sphincter; administering a second botulinum toxin dose in the pyloric region including the pyloric canal and/or the pyloric antrum; and administering a third botulinum toxin dose into the duodenum; thereby reducing the severity of at least one or more symptoms of the disorder including abdominal pain or discomfort, episodic stomach swelling, stomach pain, early fullness, abdominal bloating, early satiety, post-prandial fullness, post-prandial satiety, nausea, vomiting, acid reflux, diarrhea, problems swallowing, and combinations thereof.

Disclosed methods further comprise methods of treating gastroparesis comprising sequentially;
a. administering a botulinum toxin dose into the pyloric sphincter;
b. administering a botulinum toxin dose in the pyloric canal;
c. administering a botulinum toxin dose into the duodenum (to aid in chemical digestion of and preparation for absorption in the small intestine) during the initial or follow-up treatment; and
d. optionally injecting a botulinum toxin dose into the fundus, proximal corpus and/or the pyloric antrum, thereby reducing the severity of at least one or more symptoms of the disorder including abdominal pain or discomfort, episodic stomach swelling, stomach pain, early fullness, abdominal bloating, early satiety, post-prandial fullness, post-prandial satiety, nausea, vomiting, acid reflux, diarrhea, problems swallowing, and combinations thereof.

Neurotoxin Dosages

The neurotoxin can be administered in an amount of between about 10⁻³ U/kg and about 35 U/kg. In an embodiment, the neurotoxin is administered in an amount of between about 10⁻² U/kg and about 25 U/kg. In another embodiment, the neurotoxin is administered in an amount of between about 10⁻¹ U/kg and about 15 U/kg. In another embodiment, the neurotoxin is administered in an amount of between about 1 U/kg and about 10 U/kg. In many instances, an administration of from about 1 unit to about 300 Units of a neurotoxin, such as a botulinum type A, provides effective therapeutic relief. In an embodiment, from about 50 Units to about 400 Units of a neurotoxin, such as a botulinum type A, can be used and in another embodiment, from about 100 Units to about 300 Units of a neurotoxin, such as a botulinum type A, can be locally administered into a target tissue.

In embodiments, administration can comprise a total dose per treatment session of about 100 Units of a botulinum neurotoxin, or about 110 Units, or about 120 Units, or about 130 Units, or about 140 Units, or about 150 Units, or about 160 Units, or about 170 Units, or about 180 Units, or about 190 Units, or about 200 Units, or about 210 Units, or about 220 Units, or about 230 Units, or about 240 Units, or about 250 Units, or about 260 Units, or about 270 Units, or about 280 Units, or about 290 Units, or about 300 Units, or about 320 Units, or about 340 Units, or about 360 Units, or about 380 Units, or about 400 Units, or about 450 Units, or about 500 Units, or the like.

In embodiments, administration can comprise a total dose per treatment session of not less than 100 Units of a botulinum neurotoxin, or not less than 110 Units, or not less than 120 Units, or not less than 130 Units, or not less than 140 Units, or not less than 150 Units, or not less than 160 Units, or not less than 170 Units, or not less than 180 Units, or not less than 190 Units, or not less than 200 Units, or not less than 210 Units, or not less than 220 Units, or not less than 230 Units, or not less than 240 Units, or not less than 250 Units, or not less than 260 Units, or not less than 270 Units, or not less than 280 Units, or not less than 290 Units, or not less than 300 Units, or not less than 320 Units, or not less than 340 Units, or not less than 360 Units, or not less than 380 Units, or not less than 400 Units, or not less than 450 Units, or not less than 500 Units, or the like.

In embodiments, administration can comprise a total dose per treatment session of not more than 100 Units of a botulinum neurotoxin, or not more than 110 Units, or not more than 120 Units, or not more than 130 Units, or not more than 140 Units, or not more than 150 Units, or not more than 160 Units, or not more than 170 Units, or not more than 180 Units, or not more than 190 Units, or not more than 200 Units, or not more than 210 Units, or not more than 220 Units, or not more than 230 Units, or not more than 240 Units, or not more than 250 Units, or not more than 260 Units, or not more than 270 Units, or not more than 280 Units, or not more than 290 Units, or not more than 300 Units, or not more than 320 Units, or not more than 340 Units, or not more than 360 Units, or not more than 380 Units, or not more than 400 Units, or not more than 450 Units, or not more than 500 Units, or the like.

In embodiments, the total dose administered to the target sites in the pyloric sphincter can be, for example, about 30 Units of a botulinum neurotoxin, or about 40 Units, or about 50 Units, or about 60 Units, or about 70 Units, or about 80 Units, or about 90 Units, or about 100 Units, or about 110 Units, or about 120 Units, or about 130 Units, or about 140 Units, or about 150 Units, or about 160 Units, or about 170 Units, or about 180 Units, or about 190 Units, or about 200 Units, or about 210 Units, or about 220 Units, or about 230 Units, or about 240 Units, or about 250 Units, or about 260 Units, or about 270 Units, or about 280 Units, or about 290 Units, or about 300 Units, or the like.

In embodiments, the total dose administered to the target sites in the pyloric sphincter can be, for example, at least 30 Units of a botulinum neurotoxin, at least 40 Units, at least 50 Units, at least 60 Units, at least 70 Units, at least 80 Units, at least 90 Units, at least 100 Units, at least 110 Units, at least 120 Units, at least 130 Units, at least 140 Units, at least 150 Units, at least 160 Units, at least 170 Units, at least 180 Units, at least 190 Units, at least 200 Units, at least 210 Units, at least 220 Units, at least 230 Units, at least 240 Units, at least 250 Units, at least 260 Units, at least 270 Units, at least 280 Units, at least 290 Units, at least 300 Units, or the like.

In embodiments, the total dose administered to the target sites in the pyloric sphincter can be, for example, not more than 30 Units of a botulinum neurotoxin, not more than 40 Units, not more than 50 Units, not more than 60 Units, not more than 70 Units, not more than 80 Units, not more than 90 Units, not more than 100 Units, not more than 110 Units, not more than 120 Units, not more than 130 Units, not more than 140 Units, not more than 150 Units, not more than 160 Units, not more than 170 Units, not more than 180 Units, not more than 190 Units, not more than 200 Units, not more than 210 Units, not more than 220 Units, not more than 230 Units, not more than 240 Units, not more than 250 Units, not more than 260 Units, not more than 270 Units, not more than 280 Units, not more than 290 Units, not more than 300 Units, or the like.

In embodiments, the total dose administered to the target sites in the gastric fundus and proximal corpus can be, for example, about 30 Units of a botulinum neurotoxin, or about 40 Units, or about 50 Units, or about 60 Units, or about 70 Units, or about 80 Units, or about 90 Units, or about 100 Units, or about 110 Units, or about 120 Units, or about 130 Units, or about 140 Units, or about 150 Units, or about 160 Units, or about 170 Units, or about 180 Units, or about 190 Units, or about 200 Units, or about 210 Units, or about 220 Units, or about 230 Units, or about 240 Units, or about 250 Units, or about 260 Units, or about 270 Units, or about 280 Units, or about 290 Units, or about 300 Units, or the like.

In embodiments, the total dose administered to the target sites in the gastric fundus and proximal corpus can be, for example, at least 30 Units of a botulinum neurotoxin, at least 40 Units, at least 50 Units, at least 60 Units, at least 70 Units, at least 80 Units, at least 90 Units, at least 100 Units, at least 110 Units, at least 120 Units, at least 130 Units, at least 140 Units, at least 150 Units, at least 160 Units, at least 170 Units, at least 180 Units, at least 190 Units, at least 200 Units, at least 210 Units, at least 220 Units, at least 230 Units, at least 240 Units, at least 250 Units, at least 260 Units, at least 270 Units, at least 280 Units, at least 290 Units, at least 300 Units, or the like.

In embodiments, the total dose administered to the target sites in the gastric fundus and proximal corpus can be, for example, not more than 30 Units of a botulinum neurotoxin, not more than 40 Units, not more than 50 Units, not more than 60 Units, not more than 70 Units, not more than 80 Units, not more than 90 Units, not more than 100 Units, not more than 110 Units, not more than 120 Units, not more than 130 Units, not more than 140 Units, not more than 150 Units, not more than 160 Units, not more than 170 Units, not more than 180 Units, not more than 190 Units, not more than 200 Units, not more than 210 Units, not more than 220 Units, not more than 230 Units, not more than 240 Units, not more than 250 Units, not more than 260 Units, not more than 270 Units, not more than 280 Units, not more than 290 Units, not more than 300 Units, or the like.

In embodiments, the total dose administered to the target sites in the pyloric antrum and/or pyloric canal and/or duodenum can be, for example, about 30 Units of a botulinum neurotoxin, or about 40 Units, or about 50 Units, or about 60 Units, or about 70 Units, or about 80 Units, or about 90 Units, or about 100 Units, or about 110 Units, or about 120 Units, or about 130 Units, or about 140 Units, or about 150 Units, or about 160 Units, or about 170 Units, or about 180 Units, or about 190 Units, or about 200 Units, or about 210 Units, or about 220 Units, or about 230 Units, or about 240 Units, or about 250 Units, or about 260 Units, or about 270 Units, or about 280 Units, or about 290 Units, or about 300 Units, or the like.

In embodiments, the total dose administered to the target sites in the pyloric antrum and/or pyloric canal and/or duodenum can be, for example, at least 30 Units of a botulinum neurotoxin, at least 40 Units, at least 50 Units, at least 60 Units, at least 70 Units, at least 80 Units, at least 90 Units, at least 100 Units, at least 110 Units, at least 120 Units, at least 130 Units, at least 140 Units, at least 150 Units, at least 160 Units, at least 170 Units, at least 180 Units, at least 190 Units, at least 200 Units, at least 210 Units, at least 220 Units, at least 230 Units, at least 240 Units, at least 250 Units, at least 260 Units, at least 270 Units, at least 280 Units, at least 290 Units, at least 300 Units, or the like.

In embodiments, the total dose administered to the target sites in the pyloric antrum and/or pyloric canal and/or duodenum can be, for example, not more than 30 Units of a botulinum neurotoxin, not more than 40 Units, not more than 50 Units, not more than 60 Units, not more than 70 Units, not more than 80 Units, not more than 90 Units, not more than 100 Units, not more than 110 Units, not more than 120 Units, not more than 130 Units, not more than 140 Units, not more than 150 Units, not more than 160 Units, not more than 170 Units, not more than 180 Units, not more than 190 Units, not more than 200 Units, not more than 210 Units, not more than 220 Units, not more than 230 Units, not more than 240 Units, not more than 250 Units, not more than 260 Units, not more than 270 Units, not more than 280 Units, not more than 290 Units, not more than 300 Units, or the like.

In embodiments, administration can comprise a total dose per year of not more than 800 Units of a neurotoxin, for example botulinum type A neurotoxin, or not more than 900 Units, or not more than 1000 Units, or not more than 1200 Units, or not more than 1400 Units, or the like.

In embodiments, the dose of the neurotoxin is expressed in protein amount or concentration. For example, in embodiments the neurotoxin can be administered in an amount of between about 0.2 ng and 20 ng. In an embodiment, the neurotoxin is administered in an amount of between about 0.3 ng and 19 ng, about 0.4 ng and 18 ng, about 0.5 ng and 17 ng, about 0.6 ng and 16 ng, about 0.7 ng and 15 ng, about 0.8 ng and 14 ng, about 0.9 ng and 13 ng, about 1.0 ng and 12 ng, about 1.5 ng and 11 ng, about 2 ng and 10 ng, about 5 ng and 7 ng, and the like, into a target tissue such as a muscle.

Ultimately, however, both the quantity of toxin administered and the frequency of its administration will be at the discretion of the physician responsible for the treatment and will be commensurate with questions of safety and the effects produced by the toxin.

Disclosed embodiments comprise treatments that can be repeated. For example, a repeat treatment can be performed when the patient begins to experience symptoms of gastroparesis. However, preferred embodiments comprise repeating the treatment prior to the return of symptoms. Therefore, disclosed embodiments comprise repeating the treatment, for example, after 6 weeks, 8 weeks, 10 weeks, 12 weeks, 14 weeks, 16 weeks, 18 weeks, 20 weeks, 22 weeks, 24 weeks, or more. Repeat treatments can comprise administration sites that differ from the administration sites used in a prior treatment.

A controlled release system can be used in the embodiments described herein to deliver a neurotoxin in vivo at a predetermined rate over a specific time period. A controlled release system can be comprised of a neurotoxin incorporated into a carrier. The carrier can be a polymer or a bio-ceramic material. The controlled release system can be injected, inserted or implanted into a selected location of a patient's body and reside therein for a prolonged period during which the neurotoxin is released by the implant in a manner and at a concentration which provides a desired therapeutic efficacy.

Polymeric materials can release neurotoxins due to diffusion, chemical reaction or solvent activation, as well as upon influence by magnetic, ultrasound or temperature change factors. Diffusion can be from a reservoir or matrix. Chemical control can be due to polymer degradation or cleavage of the drug from the polymer. Solvent activation can involve swelling of the polymer or an osmotic effect.

A kit for practicing disclosed embodiments is also encompassed by the present disclosure. The kit can comprise a 30 gauge or smaller needle and a corresponding syringe. The kit can also comprise a Clostridial neurotoxin composition, such as a botulinum type A toxin composition. The neurotoxin composition may be provided in the syringe. The composition is injectable through the needle. The kits are designed in various forms based the sizes of the syringe and the needles and the volume of the injectable composition(s) contained therein, which in turn are based on the specific deficiencies the kits are designed to treat.

### EXAMPLES

The following non-limiting Examples are provided for illustrative purposes only in order to facilitate a more complete understanding of representative embodiments. This example should not be construed to limit any of the embodiments described in the present specification.

### Example 1

### Treatment of Gastroparesis

A gastroparesis patient is treated via endoscopic guided injection of 50 U total of botulinum type A into 5 injection sites spaced circumferentially in the pyloric sphincter, and 100 U total of botulinum type A into 30 injection sites on the fundus and proximal corpus (10 mm apart). The patient reports decreases in vomiting and acid reflux for 8 weeks following the treatment.

### Example 2

### Treatment of Gastroparesis

A gastroparesis patient is treated via endoscopic guided injection of 80 U total of botulinum type E into 3 injection sites circumferentially (8 mm apart) in the pyloric sphincter, and 120 U total of botulinum type A into 20 injection sites on the fundus and proximal corpus (spaced 10 mm apart). The patient reports a decrease in abdominal pain for 10 weeks following the treatment.

### Example 3

### Treatment of Gastroparesis

A gastroparesis patient is treated via endoscopic guided injection of 100 U total of botulinum type A into 7 injection sites circumferentially in the pyloric sphincter, and 100 U total of botulinum type A into 30 injection sites (10 mm apart) on the fundus and proximal corpus. The patient reports a decrease in bloating for 16 weeks following the treatment.

### Example 4

### Treatment of Gastroparesis

A gastroparesis patient is treated via endoscopic guided injection of 160 U total of botulinum type A into 5 injection sites circumferentially in the pyloric sphincter, and 80 U total of botulinum type A into 20 injection sites (10 mm apart) on the fundus and proximal corpus. The patient reports a decrease in bloating for 18 weeks following the treatment.

### Example 5

### Treatment of Gastroparesis

A gastroparesis patient is treated via endoscopic guided injection of 50 U total of botulinum type A into 5 injection sites spaced circumferentially in the pyloric sphincter, 50 U of botulinum type A into 8 injection sites on the pyloric antrum and the pyloric canal (4 injections to each), and 100 U total of botulinum type A into 30 injection sites on the fundus and proximal corpus. The patient reports decreases in vomiting and acid reflux for 8 weeks following the treatment.

### Example 6

### Treatment of Gastroparesis

A gastroparesis patient is treated via endoscopic guided injection of 80 U total of botulinum type A into 5 injection sites spaced circumferentially in the pyloric sphincter, 30 U of botulinum type A into 8 injection sites on the pyloric antrum, and 70 U total of botulinum type A into 30 injection sites on the fundus and proximal corpus (10 mm apart). The patient reports decreases in abdominal pain and nausea.

### Example 7

### Treatment of Gastroparesis

A gastroparesis patient is treated via endoscopic guided injection of 80 U total of botulinum type A into 7 injection sites spaced circumferentially in the pyloric sphincter, 40 U of botulinum type A into 8 injection sites on the pyloric canal, and 120 U total of botulinum type A into 30 injection sites on the fundus and proximal corpus (10 mm apart). The patient reports decreases in bloating and diarrhea for 12 weeks following the treatment.

### Example 8

### Treatment of Gastroparesis

A gastroparesis patient is treated via endoscopic guided injection of 50 U total of botulinum type B into 5 injection sites spaced circumferentially in the pyloric sphincter, 50 U of botulinum type A into 8 injection sites on the pyloric antrum and the pyloric canal (4 injections to each), and 100 U total of botulinum type E into 30 injection sites on the fundus and proximal corpus (10 mm apart). The patient reports decreases in abdominal pain and bloating for 6 weeks following the treatment.

### Example 9

### Treatment of Gastroparesis

A gastroparesis patient is treated via endoscopic guided injection of 50 U total of botulinum type F into 5 injection sites spaced circumferentially in the pyloric sphincter, 50 U of botulinum type E into 8 injection sites on the pyloric antrum, and 100 U total of botulinum type A into 30 injection sites on the fundus and proximal corpus (10 mm apart). The patient reports a decrease in bloating for 8 weeks following the treatment.

### Example 10

### Treatment of Gastroparesis

A gastroparesis patient is treated via endoscopic guided injection of 50 U total of botulinum type F into 6 injection sites spaced circumferentially in the pyloric sphincter, 60 U of botulinum type E into 9 injection sites on the pyloric canal, and 90 U total of botulinum type B into 30 injection sites on the fundus and proximal corpus (10 mm apart). The patient reports a decrease in acid reflux for 14 weeks following the treatment.

### Example 11

### Treatment of Gastroparesis

A gastroparesis patient is treated via endoscopic guided injection of 100 U total of botulinum type A into 5 injection sites spaced circumferentially in the pyloric sphincter, 50 U of botulinum type A into 8 injection sites on the pyloric antrum and the pyloric canal (4 injections to each), and 100 U total of botulinum type A into 30 injection sites on the duodenum. The patient reports decreases in vomiting and acid reflux for 8 weeks following the treatment.

### Example 12

### Treatment of Gastroparesis

A gastroparesis patient is treated via endoscopic guided injection of 120 U total of botulinum type A into 5 injection sites spaced circumferentially in the pyloric sphincter, 30 U of botulinum type A into 8 injection sites on the pyloric antrum, and 70 U total of botulinum type A into 10 injection sites on the duodenum. The patient reports decreases in abdominal pain and nausea.

### Example 13

### Treatment of Gastroparesis

A gastroparesis patient is treated via endoscopic guided injection of 160 U total of botulinum type A into 7 injection sites spaced circumferentially in the pyloric sphincter, 40 U of botulinum type A into 8 injection sites on the pyloric canal, and 100 U total of botulinum type A into 15 injection sites on the duodenum. The patient reports decreases in bloating and diarrhea for 12 weeks following the treatment.

### Example 14

### Treatment of Gastroparesis

A gastroparesis patient is treated via endoscopic guided injection of 150 U total of botulinum type B into 5 injection sites spaced circumferentially in the pyloric sphincter, 50 U of botulinum type A into 8 injection sites on the pyloric antrum and the pyloric canal (4 injections to each), and 100 U total of botulinum type E into 20 injection sites on the duodenum. The patient reports decreases in abdominal pain and bloating for 6 weeks following the treatment.

### Example 15

### Treatment of Gastroparesis

A gastroparesis patient is treated via endoscopic guided injection of 200 U total of botulinum type F into 5 injection sites spaced circumferentially in the pyloric sphincter, 50 U of botulinum type E into 8 injection sites on the pyloric canal, and 100 U total of botulinum type A into 20 injection sites on the duodenum. The patient reports a decrease in bloating for 8 weeks following the treatment.

### Example 16

### Treatment of Gastroparesis

A gastroparesis patient is treated via endoscopic guided injection of 150 U total of botulinum type F into 6 injection sites spaced circumferentially in the pyloric sphincter, 60 U of botulinum type E into 9 injection sites on the pyloric canal, and 90 U total of botulinum type B into 18 injection sites on the duodenum. The patient reports a decrease in acid reflux for 14 weeks following the treatment.

### Example 17

### Treatment of Gastroparesis

A gastroparesis patient is treated via endoscopic guided injection of 150 U total of botulinum type F into 6 injection sites spaced circumferentially in the pyloric sphincter, then 60 U of botulinum type E into 9 injection sites on the pyloric canal, then 90 U total of botulinum type B into 30 injection sites on the duodenum.

A follow-up treatment is performed two weeks after the initial treatment. During the follow-up treatment, 70 U of botulinum type A is administered to 10 injection sites on the fundus.

The patient reports a decrease in nausea for 14 weeks following the treatment.

### Example 18

### Treatment of Gastroparesis

A gastroparesis patient is treated via endoscopic guided injection of 180 U total of botulinum type A into 8 injection sites spaced circumferentially in the pyloric sphincter, then 60 U of botulinum type A into 6 injection sites on the pyloric canal, then 90 U total of botulinum type A into 30 injection sites on the duodenum.

A follow-up treatment is performed two weeks after the initial treatment. During the follow-up treatment, 50 U of botulinum type A is administered to 16 injection sites on the fundus and proximal corpus.

The patient reports a decrease in vomiting for 10 weeks following the treatment.

### Example 19

### Treatment of Gastroparesis

A gastroparesis patient is treated via endoscopic guided injection of 120 U total of botulinum type B into 12 injection sites spaced circumferentially in the pyloric sphincter, then 40 U of botulinum type A into 8 injection sites in the pyloric canal, then 60 U total of botulinum type A into 20 injection sites on the duodenum.

A follow-up treatment is performed three weeks after the initial treatment. During the follow-up treatment, 50 U of botulinum type B is administered to 10 injection sites on the fundus and proximal corpus.

The patient reports a decrease in stomach pain for 20 weeks following the treatment.

### Example 20

### Treatment of Gastroparesis

A gastroparesis patient is treated via endoscopic guided injection of 140 U total of botulinum type E into 16 injection sites spaced circumferentially in the pyloric sphincter, then 40 U of botulinum type E into 8 injection sites in the pyloric canal, then 60 U total of botulinum type E into 20 injection sites on the duodenum.

A follow-up treatment is performed three weeks after the initial treatment. During the follow-up treatment, 80 U of botulinum type E is administered to 20 injection sites on the fundus and proximal corpus.

The patient reports a decrease in episodic stomach swelling for 14 weeks following the treatment.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present disclosure, which is defined solely by the claims. Accordingly, embodiments of the present disclosure are not limited to those precisely as shown and described.

Certain embodiments are described herein, comprising the best mode known to the inventor for carrying out the methods and devices described herein. Of course, variations on these described embodiments will become apparent to those of ordinary skill in the art upon reading the foregoing description. Accordingly, this disclosure comprises all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

Groupings of alternative embodiments, elements, or steps of the present disclosure are not to be construed as limitations. Each group member may be referred to and claimed individually or in any combination with other group members disclosed herein. It is anticipated that one or more members of a group may be comprised in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the disclosure are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope otherwise claimed. No language in the present specification should be construed as indicating any non-claimed element essential to the practice of embodiments disclosed herein.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present disclosure so claimed are inherently or expressly described and enabled herein.

1) A method of treating gastroparesis comprising the steps of;
administering a first botulinum toxin into the pyloric sphincter; and
administering a second botulinum toxin into the fundus and proximal corpus;
thereby reducing the severity of at least one symptom of the disorder.

2) The method of claim 1, wherein at least one of said first and second botulinum toxins comprise botulinum toxin type A.

3) The method of claim 2, wherein at least one of said first and second botulinum toxins comprise native immunotype A.

4) The method of claim 1, wherein at least one of said first and second botulinum toxins comprise botulinum toxin type B, C, E, or F.

5) The method of claim 4, wherein at least one of said first and second botulinum toxins comprise botulinum toxin type B.

6) The method of claim 4, wherein at least one of said first and second botulinum toxins comprise botulinum toxin type C.

7) The method of claim 4, wherein at least one of said first and second botulinum toxins comprise botulinum toxin type E.

8) The method of claim 4, wherein at least one of said first and second botulinum toxins comprise botulinum toxin type F.

9) The method of claims 2-8, wherein at least one of said first and second botulinum toxins are administered by injection.

10) The method of claim 9, wherein said gastroparesis comprises idiopathic gastroparesis.

11) The method of claim 9, wherein said gastroparesis comprises diabetic gastroparesis.

12) The method of claim 9, wherein said treating comprises reducing a symptom of gastroparesis, said symptom comprising at least one of swollen stomach, gassiness, chronic abdominal pain or discomfort, episodic stomach swelling, stomach pain, early fullness, abdominal bloating, early satiety, post-prandial fullness, post-prandial satiety, nausea, vomiting, acid reflux, diarrhea, problems swallowing, and combinations thereof.

13) The method of claim 12, wherein said symptom comprises abdominal pain.

14) The method of claim 12, wherein said symptom comprises nausea.

15) The method of claim 12, wherein said symptom comprises vomiting.

16) The method of claim 12, wherein said symptom comprises diarrhea.

17) The method of claim 12, wherein said symptom comprises acid reflux.

18) The method of claim 12, wherein said symptom comprises swollen stomach.

19) The method of claim 1, wherein said first botulinum toxin is administered in an amount of between 50 and 200 Units.

20) The method of claim 1, wherein said first botulinum toxin is administered in an amount of between 75 and 150 Units.

21) The method of claim 1, wherein said second botulinum toxin is administered in an amount of between 50 and 200 Units.

22) A method of treating gastroparesis comprising the steps of;
administering a first botulinum toxin into the pyloric sphincter; and
administering a second botulinum toxin into at least one of the pyloric antrum and the pyloric canal;
administering a third botulinum toxin into the fundus and proximal corpus, thereby reducing the severity of at least one symptom of the disorder.

23) The method of claim 22, wherein at least one of said first, second, and third botulinum toxins comprise botulinum toxin type A.

24) The method of claim 23, wherein at least one of said first, second, and third botulinum toxins comprise native immunotype A.

25) The method of claim 22, wherein at least one of said first, second, and third botulinum toxins comprise botulinum toxin type B, C, E, or F.

26) The method of claim 25, wherein at least one of said first, second, and third botulinum toxins comprise botulinum toxin type B.

27) The method of claim 25, wherein at least one of said first, second, and third botulinum toxins comprise botulinum toxin type C.

28) The method of claim 25, wherein at least one of said first, second, and third botulinum toxins comprise botulinum toxin type E.

29) The method of claim 25, wherein at least one of said first, second, and third botulinum toxins comprise botulinum toxin type F.

30) The method of claim 23, wherein at least one of said first, second, and third botulinum toxins are administered by injection.

31) The method of claim 30, wherein said treating comprises reducing a symptom of gastroparesis, said symptom comprising at least one of swollen stomach, gassiness, chronic abdominal pain or discomfort, episodic stomach swelling, stomach pain, early fullness, abdominal bloating, early satiety, post-prandial fullness, post-prandial satiety, nausea, vomiting, acid reflux, diarrhea, problems swallowing, and combinations thereof.

32) The method of claim 31, wherein said symptom comprises abdominal pain.

33) The method of claim 31, wherein said symptom comprises nausea.

34) The method of claim 31, wherein said symptom comprises vomiting.

35) The method of claim 31, wherein said symptom comprises diarrhea.

36) The method of claim 31, wherein said symptom comprises acid reflux.

37) The method of claim 22, wherein said first botulinum toxin is administered in an amount of between 50 and 200 Units.

38) The method of claim 22, wherein said second botulinum toxin is administered in an amount of between 50 and 200 Units.

39) The method of claim 22, wherein said third botulinum toxin is administered in an amount of between 50 and 200 Units.

40) A method of treating gastroparesis comprising the steps of;
administering a first botulinum toxin into the pyloric sphincter; and
administering a second botulinum toxin into at least one of the pyloric antrum and the pyloric canal;
administering a third botulinum toxin into the duodenum,
thereby reducing the severity of at least one symptom of the disorder.

41) The method of claim 40, wherein at least one of said first, second, and third botulinum toxins comprise botulinum toxin type A.

42) The method of claim 41, wherein at least one of said first, second, and third botulinum toxins comprise native immunotype A.

43) The method of claim 40, wherein at least one of said first, second, and third botulinum toxins comprise botulinum toxin type B, C, E, or F.

44) The method of claim 43, wherein at least one of said first, second, and third botulinum toxins comprise botulinum toxin type B.

45) The method of claim 43, wherein at least one of said first, second, and third botulinum toxins comprise botulinum toxin type C.

46) The method of claim 43, wherein at least one of said first, second, and third botulinum toxins comprise botulinum toxin type E.

47) The method of claim 43, wherein at least one of said first, second, and third botulinum toxins comprise botulinum toxin type F.

48) The method of claim 41, wherein at least one of said first, second, and third botulinum toxins are administered by injection.

49) The method of claim 48, wherein said treating comprises reducing a symptom of gastroparesis, said symptom comprising at least one of swollen stomach, gassiness, chronic abdominal pain or discomfort, episodic stomach swelling, stomach pain, early fullness, abdominal bloating, early satiety, post-prandial fullness, post-prandial satiety, nausea, vomiting, acid reflux, diarrhea, problems swallowing, and combinations thereof.

50) The method of claim 49, wherein said symptom comprises abdominal pain.

51) The method of claim 49, wherein said symptom comprises nausea.

52) The method of claim 49, wherein said symptom comprises vomiting.

53) The method of claim 49, wherein said symptom comprises diarrhea.

54) The method of claim 49, wherein said symptom comprises acid reflux.

55) The method of claim 40, wherein said first botulinum toxin is administered in an amount of between 50 and 200 Units.

56) The method of claim 40, wherein said second botulinum toxin is administered in an amount of between 50 and 200 Units.

57) The method of claim 40, wherein said third botulinum toxin is administered in an amount of between 50 and 200 Units.

58) A method of treating gastroparesis comprising the steps of;
a) administering a first botulinum toxin into the pyloric sphincter;
b) administering a second botulinum toxin into the pyloric canal;
c) administering a third botulinum toxin into the duodenum;
d) thereby reducing the severity of at least one symptom of the disorder.

59) The method of claim 58, further comprising administering a fourth botulinum toxin into at least one of the fundus, proximal corpus, and pyloric antrum.

60) The method of claim 58 or 59, wherein at least one of said first, second, third, and fourth botulinum toxins comprise botulinum toxin type A.

61) The method of claim 60, wherein at least one of said first, second, third and fourth botulinum toxins comprise native immunotype A.

62) The method of claim 58 or 59, wherein at least one of said first, second, third, and fourth botulinum toxins comprise botulinum toxin type B, C, E, or F.

63) The method of claim 62, wherein at least one of said first, second, third, and fourth botulinum toxins comprise botulinum toxin type B.

64) The method of claim 62, wherein at least one of said first, second, third, and fourth botulinum toxins comprise botulinum toxin type C.

65) The method of claim 62, wherein at least one of said first, second, third, and fourth botulinum toxins comprise botulinum toxin type E.

66) The method of claim 62, wherein at least one of said first, second, third, and fourth botulinum toxins comprise botulinum toxin type F.

67) The method of claim 58 or 59, wherein at least one of said first, second, third, and fourth botulinum toxins are administered by injection.

68) The method of claim 58 or 59, wherein said treating comprises reducing a symptom of gastroparesis, said symptom comprising at least one of swollen stomach, gassiness, chronic abdominal pain or discomfort, episodic stomach swelling, stomach pain, early fullness, abdominal bloating, early satiety, post-prandial fullness, post-prandial satiety, nausea, vomiting, acid reflux, diarrhea, problems swallowing, and combinations thereof.

69) The method of claim 68, wherein said symptom comprises abdominal pain.

70) The method of claim 68, wherein said symptom comprises nausea.

71) The method of claim 68, wherein said symptom comprises vomiting.

72) The method of claim 68, wherein said symptom comprises diarrhea.

73) The method of claim 68, wherein said symptom comprises acid reflux.

74) The method of claim 68, wherein said first botulinum toxin is administered in an amount of between 50 and 200 Units.

75) The method of claim 68, wherein said second botulinum toxin is administered in an amount of between 50 and 200 Units.

76) The method of claim 68, wherein said third botulinum toxin is administered in an amount of between 50 and 200 Units.

77) A method for comparing the efficacy and safety of two different Botulinum toxins, comprising:
measuring a reduction of a gastroparesis symptom of an individual resulting from administration of a first botulinum neurotoxin;
measuring a reduction of a gastroparesis symptom of an individual resulting from administration of a second botulinum neurotoxin; and
comparing the reduction in symptoms to determine a difference between the first botulinum neurotoxin and the second botulinum neurotoxin.

78) A method of demonstrating improvement in gastric emptying and reduction in pyloric hypertonicity demonstrated on antro-pyloric manometry in patients with gastroparesis.
